(19) United States
(12) Patent Application Publication
(10) Pub. No.: US 2004/0111067 A1
(43) Pub. Date: Jun. 10, 2004

(54) Safety cannula

(76) Inventor: Fritz Kirchhofer, Sumiswald (CH)

Correspondence Address: David E. Bruhn, DORSEY & WHITNEY LLP, Intellectual Property Department, 50 South Sixth Street, Suite 1500, Minneapolis, MN, 55402-1498, US

(21) Appl. No.: 10/717,766

(22) Filed: Nov. 20, 2003

(30) Foreign Application Priority Data
Nov. 21, 2002 (DE) 102 54 442.5
Nov. 21, 2002 (DE) 102 54 441.7

(51) Int. Cl. A61M 5/00

(52) U.S. Cl. 604/240; 604/263

(57) ABSTRACT

A cannula support including an inner thread turning in a first direction and an outer thread turning in a second, opposite direction, and a system including the cannula support and a protective cap having an inner thread which can engage with the outer thread of the cannula support. In one embodiment, the invention is a cannula support including a plug-on or push-on protective cap, wherein a latching element is provided on the cannula support and a corresponding element is provided on the protective cap in order to create a latching connection, and encompasses a method for securing or covering a cannula carried by the cannular support, the cannula temporarily covered when the protective cap is arranged on the cannula support but the latching and corresponding elements are not engaged with each other, and permanently covered when the latching and corresponding elements are engaged with each other.

### CROSS-REFERENCE TO RELATED APPLICATION

The present application claims priority of German Applications No. 102 54 441.7 and 102 54 442.5, both filed Nov. 21, 2002, the contents of which are hereby incorporated by reference.

### BACKGROUND

The present invention relates to injection devices and needles or cannulae for such devices, and to devices for covering and protecting needles or cannulae. More particularly, the present invention relates to covers, shields and protectors for needles, including safety covers or shields for needles and cannulae. In particular, the present invention relates to a needle or cannula support which holds a cannula arranged in it, the cannula being of the type suitable for use with injection devices including those known as injection pens, for example, those for administering medical substances. The invention further relates to a system comprising a cannula support and a safety cap.

Injection devices, and the needles or cannulae associated with such devices, are used to introduce substances, for example subcutaneously, into a body, in order to be able to administer particular dosages to patients. To this end, injection pens are used which contain the substances to be administered in a reservoir. For reasons of hygiene, it is common that exchangeable cannulae are releaseably attached to or plugged onto a pen in a way such that they can be removed and disposed of once they have been used once or a number of times. Connecting and removing the cannulae has, however, hitherto been awkward and incurs danger of injury for the user.

A disposable injection pen needle is known from EP 0 903 157 A2. The needle is held by a needle support. The system described in EP 0 903 157 A2 substantially consists of three separate elements, namely an outer cover, an inner cover and the needle support with a needle arranged in it: In its initial state, these three elements are nested, such that the needle is surrounded by the inner and the outer cover. The needle support can be slid onto a pen and the covers removed. For removing, the outer cover is slid onto the needle support, the needle is removed and the rear side of the needle support is latched to the inner cover, in its initial state on the opposite side of the needle support to the outer cover.

### SUMMARY

It is an object of the present invention to provide a cannula support and a system comprising a cannula support and at least one protective cap, which enable a cannula support to be easily attached to and/or removed from an injection device such as an injection pen with little risk of injury.

In one embodiment, the present invention comprises a cannula support which can support a cannula or needle and to which a protective cap for the cannula can be attached, wherein the cannula support has a thread turning in a first direction, such as for example an outer or inner thread for connecting to a pen, and a thread turning in a second, opposite direction, such as for example an inner or outer thread for connecting to a protective cap.

In one embodiment, the force required to screw the cannula support onto the pen is relatively smaller than the force required to release the threaded or screw connection between the cannula support and the protective cap. A cannula support screwed to the protective cap can thus be screwed onto the pen by turning in a first direction, which is transferred by the protective cap to the cannula support. If the cannula support is screwed far enough onto the pen that the screw connection between the pen and the cannula support, and/or a holding element, applies a force against turning the cannula support farther. This force is relatively greater than the force required to release the connection and, therefore, generates a turning movement of the screw connection between the cannula support and the protective cap. If the protective cap is turned farther, it is unscrewed from the cannula support screwed to the pen. Thus, a screw connection between the cannula support and the pen can be established by a turning movement in a single direction and, after the screw connection between the cannula support and the pen has been established, the protective cap can be unscrewed or removed from the cannula support by continued turning in the same direction.

For removing the cannula support from the pen, the protective cap can first be screwed back onto the cannula support by turning it in a second, opposite direction. When the protective cap screwed onto the cannula support is turned farther, the cannula support can be unscrewed from the pen due to the opposite direction of the screw connection between the cannula support and the pen. Replacing the cap in this way reduces the danger of injury when attaching a cannula support or a cannula to a pen, and/or when removing a cannula support or a cannula from a pen.

In one embodiment, a left-handed thread can be provided on the outside of the cannula support or on the inside of the protective cap, wherein the cannula support comprises on its inside a right-handed thread for connection to a corresponding counter, or complemetary, thread on the pen. Alternatively, the outer thread of the cannula support can be a right-handed thread cooperating with the protective cap, while the inner thread of the cannula support—cooperating with the pen—is a left-handed thread.

In one embodiment, the present invention comprises a cannula support including an inner thread turning in a first direction and an outer thread turning in a second, opposite direction, and a system including the cannula support and a protective cap having an inner thread which can engage with the outer thread of the cannula support. In one embodiment, the invention further comprises a cannula support and a plug-on or push-on protective cap, wherein a latching element is provided on the cannula support and a corresponding element is provided on the protective cap in order to create a latching connection, and encompasses a method for securing or covering a cannula carried by the cannular support, wherein the cannula is temporarily covered when the protective cap is arranged on the cannula support but the latching and corresponding elements are not engaged with each other, and wherein the cannula is permanently covered when the latching and corresponding elements are engaged with each other.

Advantageously, in some embodiments, at least one latching element for creating a latching connection between the protective cap and the cannula support can be provided on the cannula support and/or on the protective cap. The at least one latching element may be arranged such that when the cannula support is screwed onto a pen with the aid of the protective cap, the latching connection is not yet latched in, and when the cannula support is unscrewed from the pen, the protective cap screwed onto the cannula support latches. In this way, the cannula support is fixedly connected to the protective cap via the at least one latching element before the cannula support is unscrewed from the pen, preventing the cannula from falling off and being exposed.

In one advantageous embodiment, another protective cap is provided in addition to the protective cap used for screwing the cannula support onto the pen and/or unscrewing the cannula support from the pen. This second protective cap surrounds the cannula and can be removed. As described herein below, it can comprise one or more latching elements for establishing a fixed and, in some embodiments, a preferably non-releasable, connection between the cannula support and a protective cap.

In one embodiment, the present invention comprises a needle cover comprising a needle support carrying a needle, said needle support comprising a cam and an inside surface with an inside thread for coupling the support to an injection device, an inner protective cap coupled to the needle support, and an outer protective cap generally surrounding the inner protective cap and comprising a left-handed thread and a cavity on an inside surface, said needle support comprising a complementary counter thread whereby the outer protective cap and the needle support may be releaseably connected to each other, said counter thread turning in a direction generally opposite to the inside thread, said cam received in said cavity after said needle support is coupled to the injection device.

In accordance with one embodiment, a cannula support in accordance with the present invention can support a cannula or needle for administering a medical substance, and a removable protective cap can be attached to the cannula support. In accordance with the present invention, the cannula support has at least one latching element, such as one or more cams, a ring encircling an outer side of the cannula support, recesses and/or elevations on the surface, or the like. The at least one latching element can hold the protective cap if the at least one latching element is latched with at least one equivalent, corresponding latching element on the protective cap, such as a complementary recess or a cam.

In one embodiment, the protective cap in its initial state can be moved or slid onto the cannula support just far enough for the at least one latching element on the cannula support to not quite latch with the corresponding latching element on the protective cap, such that the protective cap can be removed from and replaced on the cannula support once or a number of times, as long as the at least one latching element is not latched. If and/or when the cannula is to be removed and disposed of, the protective cap can be slid over the at least one latching element, such that the protective cap is fixedly connected to the cannula support by the latching connection thus created, and can no longer be removed from it. The cannula is therefore securely and permanently surrounded by the protective cap, such that there is practically no longer a risk of injury or infection from a used cannula.

In some embodiments, the at least one latching element on the cannula support can be a cam, a protruding element, a circumferential ring, an element charged in a particular direction by a spring force, a recess, a through-hole or any other suitable element which, together with a corresponding, complementary counter element on the protective cap, can enter into or establish a fixed connection which is as difficult as possible to release or is non-releasable, in particular a frictional-lock or positive-lock connection. The counter element on the protective cap may take the form of a recess or an opening in the protective cap, which the at least one latching element can latch into in order to latch and establish the non-releasable connection. It is equally possible to provide at least one latching element on the protective cap, which can latch with a corresponding counter element on the cannula support, wherein both elements—the cannula support and the protective cap—can comprise latching and corresponding counter latching elements. In some embodiments, the latching element is preferably arranged laterally, and on an outer surface of the cannula support.

The cannula support and/or the protective cap can be formed such that the protective cap can be attached, e.g., plugged and/or screwed onto, to the cannula support in such a way that it can be removed. In the case of a screw connection, at least one latching element is advantageously provided such that a fixed and preferably non-releasable latch is only created after a given number of turns. For example, in some embodiments, the protective cap can be screwed onto the cannula support by turning it twice, and can be removed by turning it twice the opposite way. However, turning the protective cap farther in the first or “on” direction, i.e., beyond two turns, causes it to latch such that the protective cap can no longer be unscrewed from the cannula support. This arrangement ensures that the cannula can be safely removed and disposed of.

In accordance with a preferred embodiment, the cannula support and/or the protective cap comprises—in addition to the at least one latching element for creating a fixed connection as described above—at least one other latching element which is advantageously arranged in front of the one latching element in the connection direction, such that the protective cap can be attached to the cannula support and latched to the front latching element, in order to hold the protective cap from unintentionally being detached from the cannula support. The front latching connection is, however, formed such that only a small holding force acts, i.e., a protective cap held on the cannula support by this latching connection can easily be removed from the cannula support, for example by turning or pulling, in order to expose the cannula. If the cannula is to be removed and disposed of, then the protective cap is slid or turned past the front latching connection until the rear latching connection provided in accordance with the invention latches. The protective cap is thus fixedly connected to the cannula support and can no longer be removed.

A cannula support in the sense of the present invention may be a disposable part and may be attached to, plugged on or screwed on an injection pen, used together with a cannula a number of times and exchanged in order to be disposed of. Equally, a cannula support in accordance with the present invention may be fixedly connected to an injection device, such that it cannot be removed or can be removed only with difficulty, whereby, in conjunction with the latching mechanism described above, a cannula or needle arranged on the injection device is securely covered for safe disposal.

A cannula support in accordance with the present invention may be releasably and/or non-releasably connected to an injection device such as an injection pen. In the case of a releasable connection, it is advantageous to design the releasable connection such that a force required to release said connection between the cannula support and the pen is smaller than the force required to release a connection between the protective cap and the cannula support. The protective cap connected to the cannula support can thus be moved or screwed in the removing direction, i.e., away from the pen, and will take the cannula support with it. Due to the weaker connection between the cannula support and the pen, this connection is released and the cannula support is removed from the pen, the needle carried by the support thereby remaining securely in the protective cap in order to avoid injuries or infection. Advantageously, the force for releasing a temporary connection between the cannula support and the protective cap, generated by another latching element, is smaller than the force required to release the cannula support from the pen.

In accordance with another aspect of the present invention, the invention relates to a method for securing or protecting a cannula held by a cannula support by using a protective cap, wherein the protective cap—in order to be removed again later from the cannula support—is only moved or slid on to the support at most to just before entering a permanent latching position, such that the permanent latching connection does not latch. Moving or sliding the protective cap to the latching position or beyond create a fixed and preferably non-releasable latching connection.

### BRIEF DESCRIPTION OF THE DRAWINGS

FIG. 1 is a cross-sectional view of a cannula support attached to an ijection pen and comprising a non-fixedly latched protective cap;

FIG. 2 depicts the arrangement shown in FIG. 1, comprising a fixedly latched protective cap;

FIG. 3 is a cross-sectional view of a cannula support screwed onto an injection pen and comprising protective cap; and

FIG. 4 is a top view of the arrangement shown in FIG. 3.

### DETAILED DESCRIPTION

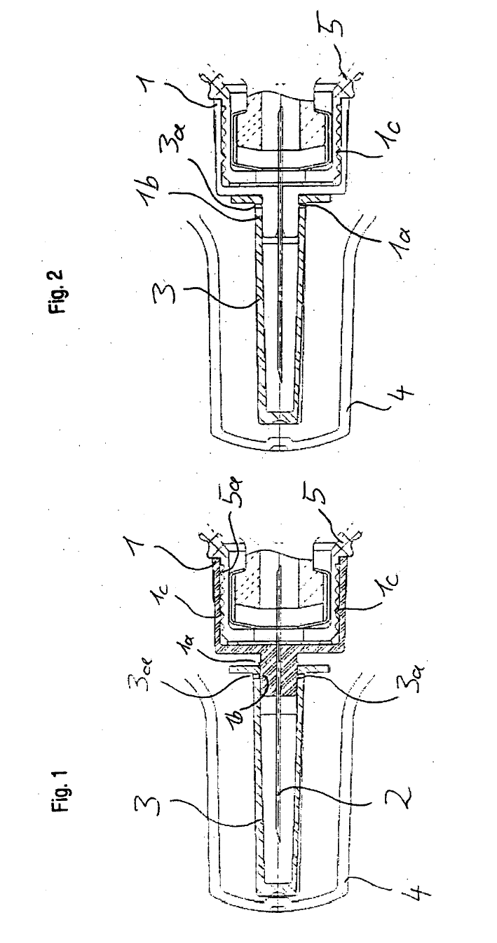

FIG. 1 shows a cannula support 1 screwed onto a pen 5, in which a cannula 2 is arranged. The cannula 2 surrounded by a protective cap 3 plugged onto the cannula support 1 and thus secured, such that the danger of and unintentional injury is reduced. The protective cap 3 comprises passages 3a in the lateral wall at its rear end, with which latching cams 1a, 1b of the cannula support 1 can engage. In the position of the protective cap 3 shown in FIG. 1, the cam 1b—smaller in comparison with the cam 1a—engages with the passage 3a and thus creates a weak latching connection, such that while the protective cap 3 is secured against unintentionally falling off of the cannula support 1, it can however be removed from the cannula support 1 by applying a small force for releasing the latching connection created by the small cam 1b. If the protective cap is only slid on up to the position shown in FIG. 1, then it can be plugged on and removed again a number of times.

If, however, the cannula 2 is to be disposed of, then the protective cap 3 is slid over the larger latching cam 1a provided on the cannula support 1 for creating a second, stronger latching connection, as shown in FIG. 2, such that a connection which is non-releasable or can only be released with difficulty is established between the protective cap 3 and the cannula support 1. The protective cap 3 can thus no longer be removed from the cannula support 1, such that the cannula 2 is permanently secured by the plugged-on protective cap 3: This can minimize the risk of injury and the cannula support 1 can be unscrewed from the pen 5.

Although FIGS. 1 and 2 show an embodiment of a pen 5 comprising an outer thread 5a with which an inner thread or a protrusion 1c of the cannula support 1 engages, a different type of connection between the pen 5 and the cannula support 1 can also be chosen, such as for example a latching connection or other releasable connecting mechanisms.

The outer protective cap or packing sleeve 4 shown in FIGS. 1 and 2 can optionally be plugged and/or screwed onto the protective cap 3, in order to protect the protective cap 3 and optionally the cannula support 1 or the front portion of the pen 5, for example from jolts or from forces acting from without.

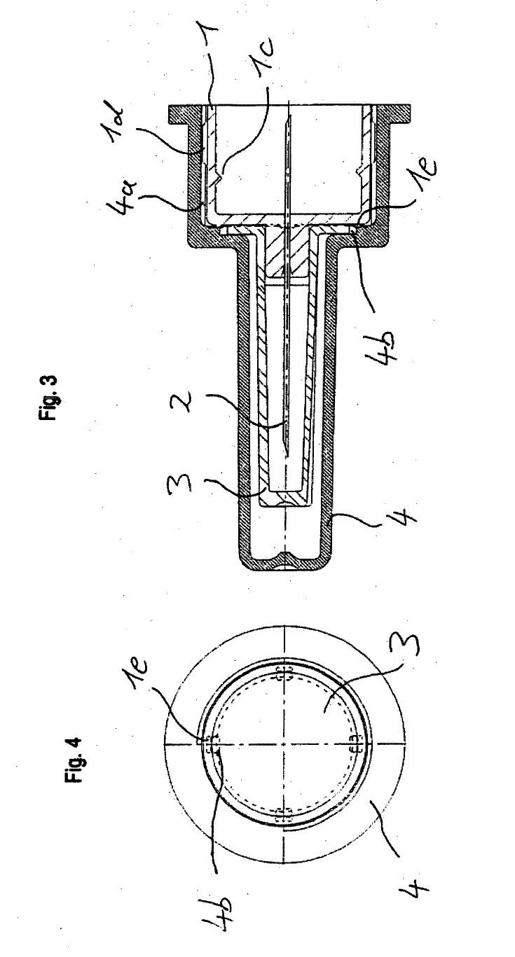

FIG. 3 shows a cross-section of a cannula support 1 in which a cannula 2 is arranged. An inner protective cap 3 is plugged onto the cannula support 1 and surrounds the cannula 2. An outer protective cap 4 surrounds the inner protective cap 3 and comprises a left-handed thread 4a on its inner side, a corresponding counter thread 1d on the cannula support 1 being screwed into said left-handed thread 4a, such that the outer protective cap 4 and the cannula support 1 are releasably connected to each other. The cannula support 1 comprises an inner thread 1c formed as a right-handed thread which serves to screw the cannula support 1 onto a pen (not shown). The cannula support 1 further comprises securing cams 1e which, prior to screwing the cannula support 1 onto the pen, do not yet engage with corresponding cavities 4b of the outer protective sleeve 4.

To be screwed onto the pen, the arrangement shown in FIG. 3 is screwed onto an outer thread of the pen corresponding to an inner thread 1c of the cannula support 1 by a right-hand turn which is transferred from the outer protective cap onto the cannula support 1. If the cannula support 1 is screwed sufficiently fixedly on the pen and/or for example the screwed-on cannula support 1 abuts a corresponding holding element of the pen, then the turning moment acting in order to screw onto the outer protective sleeve 4 is no longer completely transferred via the inner thread 4a of the outer protective sleeve 4 onto the corresponding outer thread 1d of the cannula support, wherein the outer protective sleeve 4 is released from the cannula support 1 by continuing the turning movement, such that the outer protective sleeve 4 can be unscrewed from the cannula support 1.

The cannula can then be exposed by removing the inner protective cap 3, for example in order to perform an injection.

In order to remove the cannula 2 from the pen, the outer protective sleeve 4 can be screwed back onto the cannula support 1, until for example a small turning moment is created. Through this turning moment, for example, the securing cams 1e provided on the outer side of the cannula support 1 latch into corresponding cavities 4b of the outer protective cap 4, such that a fixed connection can be established between the outer protective cap 4 and the cannula support 1. If the outer protective cap 4 is turned further, then the turning moment acting on the outer protective cap 4 is transferred onto the cannula support 1, such that the latter can be unscrewed from the pen. The latching connection created by the latching elements 1e together with the cavities in the packing sleeve 4b prevents the cannula support 1 with the possibly used cannula 2 from being able to fall out of the outer protective cap 4, such that the danger of injury is reduced.

FIG. 4 shows the arrangement shown in FIG. 3, in a top view, wherein the latching elements 1e of the cannula support are in a position in which they latch with the corresponding cavities or recesses 4b in the outer protective cover 4, in order to prevent the cannula support 1 from falling out of the outer protective cover 4.

In accordance with an embodiment not shown, the inner protective sleeve 3 shown in FIGS. 3 and 4 can comprise the latching mechanism shown in FIGS. 1 and 2 and described in this application.

In the foregoing description, embodiments of the present invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the, scope of the invention as determined by the appended claims when interpreted in accordance with the breadth they are fairly, legally, and equitably entitled.

1. A cannula support comprising a thread turning in a first direction and another thread turning in a second, generally opposite direction.

2. The cannula support as set forth in claim 1, wherein said thread turning in said first direction is an inner thread and said thread turning in said generally opposite direction is an outer thread.

3. The cannula support as set forth in claim 1, comprising at least one latching element arranged on one of the inside and outside of said cannula support.

4. A cannula system comprising a cannula support as set forth in claim 1 and a protective cap having a thread engageable with said thread turning in said second generally opposite direction.

5. The cannula system as set forth in claim 4, comprising at least one latching element arranged on one of the inside and outside of said cannula support.

6. The system as set forth in claim 5, wherein said protective cap comprises at least one complementary latching element for latching with the latching element of the cannula support.

7. The system as set forth in claim 4, wherein a second protective cap is arranged within said protective cap.

8. The system as set forth in claim 4, comprising an injection device having a thread engageable with said thread turning in the first direction.

9. A cannula support comprising a protective cap, wherein at least one latching element is provided on said cannula support and at least one corresponding latching element is provided on said protective cap, the latching elements cooperating to create a latching connection between the cannula support and the protective cap.

10. The cannula support as set forth in claim 9, wherein the latching elements cooperate to form a non-releasable latching connection.

11. The cannula support as set forth in claim 9, wherein the protective cap can be coupled to the cannula support in such a way that there is no connection between the latching elements.

12. The cannula support as set forth in claim 9, wherein one of the at least one latching element on the cannula support and the corresponding latching element on the protective cap is one of the group consisting of a cam, a circumferential ring, an element biased by a spring force, a recess and a through-hole.

13. The cannula support as set forth in claim 9, wherein the at least one latching element is provided on a lateral, outer surface of the cannula support.

14. The cannula support as set forth in claim 9, wherein at least one second latching element is provided on the cannula support and can engage with one of a corresponding counter element and the corresponding latching element on the protective cap to create a releasable connection between the cannula support and the protective cap.

15. The cannula support as set forth in claim 14, wherein the cannula support comprises connection element for connecting the cannula support to a pen.

16. The cannula support as set forth in claim 15, wherein said connection element creates a connection which requires a lesser force for releasing the connection than the force required for releasing the connection between the at least one latching element on the cannula support and the corresponding latching element on the protective cap.

17. The cannula support as set forth in claim 15, wherein said protective cap is more easily releasable from said cannula support than the cannula support is from the pen.

18. A method for covering a cannula carried by a cannula support using a cannula protecting cap, wherein the cannula support comprises a latching element, and wherein the cannula is temporarily covered when the protecting cap is coupled to the cannula support but not to the latching element and permanently covered when the protecting cap is coupled to the latching element.

19. A needle cover comprising
a needle support carrying a needle, said needle support comprising a cam and an inside surface with an inside thread for coupling the support to an injection device;
an inner protective cap coupled to the needle support; and
an outer protective cap generally surrounding the inner protective cap and comprising a left-handed thread and a cavity on an inside surface, said needle support comprising a complementary counter thread whereby the outer protective cap and the needle support may be releaseably connected to each other, said counter thread turning in a direction generally opposite to the inside thread, said cam received in said cavity after said needle support is coupled to the injection device.
